(19) United States
(12) Patent Application Publication
(10) Pub. No.: US 2020/0368404 A1
(43) Pub. Date: Nov. 26, 2020

(54) MEDICAL ASPIRATOR WITH IMPROVED SAFETY

(71) Applicants: INDUSTRIAL COOPERATION FOUNDATION CHONBUK NATIONAL UNIVERSITY, Jeollabuk-do (KR); CHONBUK NATIONAL UNIVERSITY HOSPITAL, Jeollabuk-do (KR)

(72) Inventors: Jong Kwan Park, Jeollabuk-do (KR); Jun Sung Park, Gyeonggi-do (KR); Jun Soo Park, Seoul (KR); Sun Ok Woo, Jeollabuk-do (KR)

(21) Appl. No.: 16/961,860

(22) PCT Filed: Jan. 11, 2019
(86) PCT No.: PCT/KR2019/000459

§ 371 Date: Aug. 6, 2020

(30) Foreign Application Priority Data
Jan. 12, 2018 (KR) 10-2018-0004598
Jan. 9, 2019 (KR) 10-2019-0002824
Jan. 10, 2019 (KR) 10-2019-0003271

(51) Int. Cl. A61M 1/00; A61M 39/24; A61M 39/20

(52) U.S. Cl. CPC A61M 1/0001; A61M 39/20; A61M 39/24

(57) ABSTRACT

This disclosure relates to a medical aspirator with improved safety, and, more particularly, to a medical aspirator which stores a body fluid discharged from a human body in a storage container so as not be contaminated by air and discharges the stored body fluid through a discharge pipe installed in communication with the inside to the outside of the storage container, such that body fluid can be discharged without turning the storage container upside down and the body fluid can be prevented from being ejected due to a sudden pressure difference when a discharge line is opened.

### TECHNICAL FIELD

This disclosure relates to a medical aspirator with improved safety, and, more particularly, to a medical aspirator which stores a body fluid discharged from a human body in a storage container so as not be contaminated by air and discharges the stored body fluid through a discharge pipe installed in communication with the inside to the outside of the storage container, such that body fluid can be discharged without turning the storage container upside down and the body fluid can be prevented from being ejected due to a sudden pressure difference when a discharge line is opened.

### BACKGROUND ART

In general, after a surgical operation, blood, exudate and human body fluid are collected in the tissue, and the blood or exudate collected in this way disturbs the healing of the wound to cause complications. Thus, it is needed to manage blood or exudate not to be collected in the tissue. In particular, blood or exudates drained from the operated tissues may be used to check for bleeding after surgery through quantity and color, to determine whether inflammation is caused by infection, or to monitor damage to surrounding organs (especially, large intestine and small intestine) in case of abdominal surgery.

Therefore, in order to drain blood or exudate produced after surgery, a medical aspirator commonly called ‘Hemovac’ or ‘Barovac’ or ‘Inovac’ is commonly used.

A conventional aspirator used for medical purposes has an expandable storage container, and includes an inlet pipe provided at one side of the storage container to introduce body fluid and a discharge pipe for discharging the stored body fluid. Also, in order to generate a continuous negative pressure, a number of springs are installed inside the storage container.

The conventional aspirator is mostly provided with a stopper mounted to the discharge pipe, and in order to discharge the stored body fluid, the aspirator is held with one hand and the stopper is opened with the other hand. This action compresses the aspirator while gripping the aspirator, thereby pressing the stored body fluid. In addition, if the stopper is opened while the body fluid is being pressed, the stored body fluid may be ejected due to the applied pressure. The ejection of the body fluid may be adhered to the skin or clothes of the caregiver handling the aspirator to cause contamination or hepatitis, thereby inhibiting the hygiene of the hospital room.

In addition, since the discharge pipe is located at the top, the aspirator must be turned over to discharge the stored body fluid. If the aspirator is turned upside down and the body fluid is discharged into a container, the aspirator covers the container, so it is difficult to check the amount of discharged body fluid. Also, if the discharge pipe of the aspirator comes into contact with the body fluid contained in the container, the outer surface of the aspirator may be contaminated.

In addition, if the stored body fluid is completely discharged, the discharge pipe must be closed with a stopper after the aspirator is compressed. However, since the springs built in the aspirator have a high elastic repulsive force, a user with a weak force cannot press the aspirator with one hand. Therefore, the user usually compresses the aspirator with both hands and then quickly stops the discharge pipe with one hand. However, the springs in the aspirator are elastically restored while the discharge pipe is blocked with the stopper by removing the pressing hand, which lowers the suction power of the aspirator. Thus, the aspirator cannot be used at its maximum capacity.

Korean Patent Registration No. 10-1367004 (registered on Feb. 18, 2014; hereafter, referred to as ‘Prior Literature 1’) and Korean Unexamined Patent Publication No. 10-2014-0087616 (published on Jul. 9, 2014; hereafter, referred to as ‘Prior Literature 2’) disclose a medical aspirator. Prior Literatures 1 and 2 propose a structure in which a shielding means including a check valve is installed to a discharge hole or a stopper with a backflow prevention piece is installed to the discharge hole, so that the aspirator can be easily compressed even by a weak person. However, both of Prior Literatures 1 and 2 still have a problem in that the aspirator must be turned over to discharge the body fluid stored therein. Therefore, the problems related to contamination and checking of a discharged amount during the discharge process still remain.

Therefore, there is a need for a new structure that may discharge the stored body fluid without turning over the aspirator.

In addition, a drainage tube equipped with a clamp is firstly connected to the inlet pipe formed in the conventional aspirator, and a body fluid tube fixed to the body of a patient is connected to the drainage tube.

However, in the conventional inlet pipe connection structure, when the body fluid tube is directly fixed to the drainage tube, the body fluid tube and the drainage tube may be easily separated from each other due to material characteristics thereof. That is, the body fluid tube is made of a soft material that is easy to bend in use, and the drainage tube is also made of a soft material that is easy to bend. Thus, the body fluid tube and the drainage tube are not fully fixed and may be unintentionally separated due to the movement of the patient or while the patient is moved.

If the body fluid tube and the drainage tube are separated while the body fluid is introduced into the drainage tube, the body fluid of the patient may splash or leak to the outside, which may cause a secondary infection of the medical staff or guardians.

### DISCLOSURE OF THE INVENTION

### Technical Problem

This disclosure is designed to solve the above problems, and the present disclosure is directed to providing an aspirator, which allows body fluid stored therein to be discharged without turning over the aspirator, by installing a body fluid discharge pipe, which is in communication with the outside at an inner bottom surface of a storage container, to an upper surface of the storage container by means of the aspirator having a discharge pipe.

In addition, the present disclosure is directed to providing an aspirator allowing easy and safety discharge, which may discharge body fluid without turning over the aspirator by forming the discharge pipe at a lower portion thereof, rather than an existing position, allow the discharge pipe at a lower portion to be stably placed without interfering with the bottom, and allow a user to directly check a discharge portion to prevent various medical accidents caused during the discharging process.

In addition, the present disclosure is directed to providing a medical aspirator, which may improve the connection structure between the drainage tube connected to the storage container and the body fluid tube connected to the body of a patient so that the connection structure is not easily separated even with slight pulling, and prevent medical accidents of secondary infection caused by body fluid leakage.

### Technical Solution

In one general aspect of the present disclosure, there is provided a medical aspirator with improved safety, which receives and temporarily stores human body fluid, the medical aspirator comprising: a storage container having an accommodation space formed therein; an inlet pipe coupling hole formed at an upper portion of the storage container so that an inlet pipe for introducing human body fluid is mounted thereto; a discharge pipe coupling hole formed at the upper portion of the storage container to communicate an inside and an outside of the storage container; and a discharge pipe coupled to the discharge pipe coupling hole and having a lower end located at a bottom of the accommodation space of the storage container and an upper end drawn out long to discharge body fluid stored in the storage container.

In another aspect of the present disclosure, there is also provided a medical aspirator with improved safety, which includes a storage container having a pressing plate and a bottom plate respectively provided to an upper portion and a lower portion thereof and having a storage space formed by a sealing membrane that connects the pressing plate and the bottom plate, and a spring installed between the pressing plate and the bottom plate, wherein an inlet hole for introducing body fluid and a discharge hole for discharging stored body fluid are formed at the storage container, the discharge hole is formed at the bottom plate to protrude on a bottom surface of the storage container, a supporting protrusion is further formed to protrude on the bottom surface of the bottom plate for balancing the storage container, and the discharge hole further includes a discharge tube configured to extend out of the storage container.

In another aspect of the present disclosure, there is also provided a medical aspirator with improved safety, which includes a storage container having a pressing plate and a bottom plate respectively provided to an upper portion and a lower portion thereof and having a storage space formed by a sealing membrane that connects the pressing plate and the bottom plate, and a spring installed between the pressing plate and the bottom plate, wherein an inlet hole for introducing body fluid is formed at the pressing plate or the bottom plate of the storage container, a soft drainage tube having a clamp is connected to the inlet hole, and a connection portion is formed at the drainage tube so that a soft body fluid tube fixed to the body of a patient is connected thereto, and a hard primary connection tube is firstly coupled to the connection portion between the connection portion and the body fluid tube, and a soft secondary connection tube is coupled to the primary connection tube so as to be connected to the body fluid tube.

### Advantageous Effects

In the present disclosure, since the discharge pipe is installed to the aspirator to extend to the inner bottom of the aspirator, the stored body fluid may be discharged without turning the aspirator upside down. Also, since the discharging direction may be adjusted by the discharge pipe, it is possible to prevent contamination by body fluid ejection, different from the conventional case where the body fluid is directly discharged from the discharge hole. Also, it is possible to prevent the outside of the aspirator from being contaminated due to the discharged body fluid.

In addition, in the present disclosure, since the discharge hole of the aspirator is formed at the lower portion of the storage container, the body fluid may be simply discharged without turning over the aspirator. Also, even if the discharge hole protrudes to the lower portion of the storage container, since a supporting protrusion is further formed so that the discharge hole is spaced apart from the ground, the storage container may maintain the balance without interference of the discharge hole and be stably supported on the ground.

In addition, in the present disclosure, a hard primary connection tube and a soft secondary connection tube with a strong tightening force are used in the connection structure of the body fluid tube and the drainage tube such that the body fluid tube and the drainage tube are not unintentionally separated, thereby blocking leakage of the body fluid at the connection portion and preventing medical accidents such as secondary infection. Also, since the coupling strength is enhanced by the material characteristics and insertion structure of the primary connection tube and secondary connection tube, the aspirator of the present disclosure may be easily used without complexity caused by the coupling.

### BRIEF DESCRIPTION OF THE DRAWINGS

FIG. 1 is a perspective view showing an aspirator having a discharge pipe according to the first embodiment of the present disclosure.

FIGS. 2A and 2B are sectional views showing an example where a discharge pipe coupling hole and a discharge pipe formed at a pressing plate are coupled according to the first embodiment of the present disclosure.

FIGS. 3A to 3C are a front view and plan views schematically showing that one spring or three springs are installed according to embodiments of the present disclosure.

FIGS. 4A and 4B are schematic views showing auxiliary members coupled to the discharge pipe according to embodiments of the present disclosure.

FIG. 5 is a perspective view showing an example where a perforated hole is formed in a circular support wall of a bottom plate according to an embodiment of the present disclosure.

FIGS. 6A to 6C are a plan view and vertical sectional views showing a bottom plate having a placing groove according to an embodiment of the present disclosure.

FIG. 7 is a sectional view showing a main part of the discharge pipe having an introduction hole formed at a side surface thereof according to an embodiment of the present disclosure.

FIG. 8 is a perspective view showing an aspirator having a discharge pipe according to another embodiment of the present disclosure.

FIG. 9 is a diagram showing an aspirator according to the second embodiment of the present disclosure.

FIG. 10 is a bottom perspective view showing the aspirator according to the second embodiment of the present disclosure.

FIG. 11 is a bottom view showing the aspirator according to the second embodiment of the present disclosure.

FIG. 12 is a diagram showing that a supporting protrusion employed at the second embodiment of the present disclosure includes a main support portion and a sub support portion.

FIG. 13 is a diagram showing that the supporting protrusion employed at the second embodiment of the present disclosure includes a front support portion and a rear support portion.

FIGS. 14 and 15 are diagrams showing an embodiment in which the rear support portion has an adjustable support height in the supporting protrusion employed at the second embodiment.

FIG. 16 is a diagram showing the entire aspirator of the present disclosure even with a connection configuration of the drainage tube and the body fluid tube.

FIG. 17 is an enlarged view showing a coupling structure of a primary connection tube and a secondary connection tube of the present disclosure.

FIG. 18 is a sectional view showing the primary connection tube of the present disclosure.

FIG. 19 is a diagram showing an embodiment in which a first wedge portion is further formed at the primary connection tube of the present disclosure.

FIG. 20 is a diagram showing other embodiments of the primary connection tube of the present disclosure as (A) and (B) depending on its shape.

FIG. 21 is a diagram showing an embodiment in which a second wedge portion is further formed at the secondary connection tube of the present disclosure.

FIG. 22 is a diagram showing another embodiment in which the primary connection tube and the secondary connection tube of the present disclosure are integrally coupled by a coupling groove and a coupling protrusion.

### MODE FOR CARRYING OUT THE INVENTION

Hereinafter, a preferred embodiment of the present disclosure will be described in detail with reference to the accompanying drawings. Also, in the present disclosure, if it is determined that a detailed description of the related known function or configuration may unnecessarily obscure the subject matter of the present disclosure, the detailed description will be omitted.

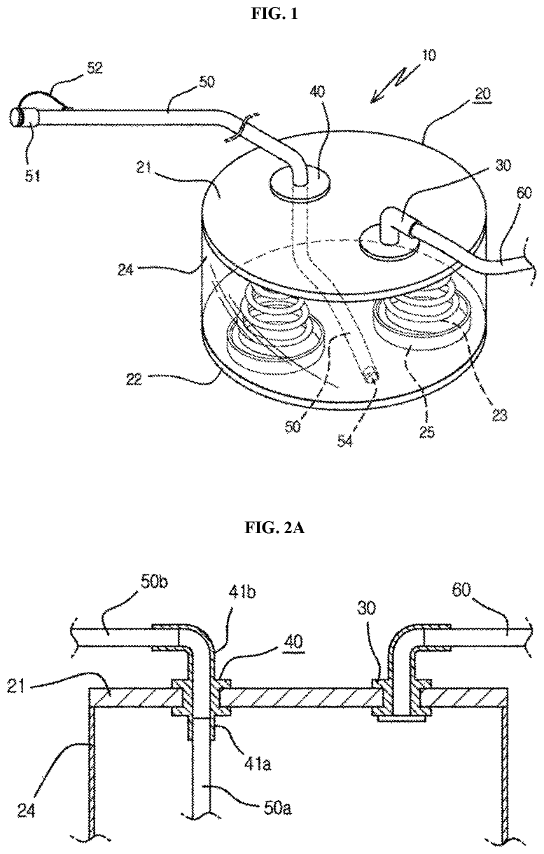

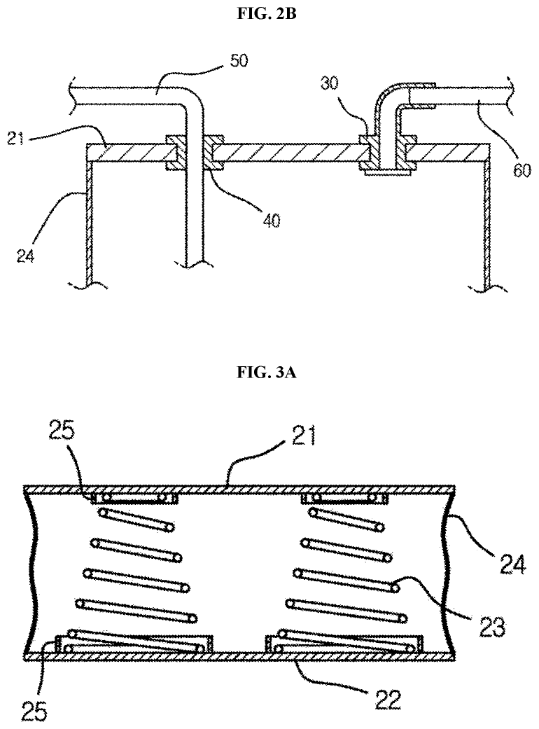

FIG. 1 is a diagram showing an aspirator according to an embodiment of the present disclosure, and FIGS. 2A and 2B are schematic sectional views showing a state where a discharge pipe and a discharge pipe coupling hole are coupled.

Referring to the figures, an aspirator 10 according to the present disclosure includes a storage container 20 having an accommodation space formed therein, an inlet pipe coupling hole 30 and a discharge pipe coupling hole 40 formed at an upper portion of the storage container, and a discharge pipe 50 mounted to the discharge pipe coupling hole.

The storage container 20 is a container configured to be shrinkable or expandable by elastically deformation so that the volume of the accommodation space may be changed. The storage container 20 according to an embodiment of the present disclosure includes a pressing plate 21 at an upper portion, a bottom plate 22 at a lower portion, and a sealing membrane 24 connecting edges of the pressing plate and the bottom plate.

As shown in FIGS. 1 and 2A, the pressing plate 21 has an inlet pipe coupling hole 30 to which an inlet pipe 60 for introducing body fluid from the human body and transporting the body fluid into the accommodation space inside the storage container is coupled. The inlet pipe coupling hole 30 is a flow path formed therein, and may protrude upward from a surface of the pressing plate 21, preferably vertically from the pressing plate, to be vertically coupled to the inlet pipe. Alternatively, as shown in the figures, the inlet pipe coupling hole 30 may be bent into a “L” shape and horizontally coupled to the inlet pipe 60 so that the transported body fluid is stored inside the storage container. At this time, a check valve 31 may be mounted to the inlet pipe coupling hole 30 so that the body fluid transported through the inlet pipe 60 is introduced into the accommodation space of the storage container but the body fluid stored in the accommodation space of the storage container 20 is blocked not to be discharged to the outside through the inlet pipe coupling hole 30 and the inlet hole.

In addition, a discharge pipe coupling hole 40 may be further formed at the pressing plate 21, and a discharge pipe 50 may be mounted to the discharge pipe coupling hole. Generally, a discharge hole, which is a perforated hole, is provided to an existing aspirator to discharge the body fluid stored therein by opening or closing the discharge hole. However, in the present disclosure, the discharge pipe coupling hole 40 is formed at the pressing plate 21, and the discharge pipe 50 is water-tightly coupled to the discharge pipe coupling hole 40 so that the body fluid is discharged through the discharge pipe, rather than the discharge hole that is a perforated hole. At this time, as shown in FIG. 2A, the discharge pipe coupling hole 40 may be fixed to the pressing plate 21, protrusions 41a, 41b having flow paths therein protrude toward the inner accommodation space at a lower portion and the outer side at an upper portion, and an internal discharge pipe 50a and an external discharge pipe 50b may be coupled to the protrusion. Alternatively, as shown in FIG. 2B, the discharge pipe coupling hole 40 may be provided in the form of a perforated hole so that the discharge pipe 50 is inserted therein, in a water-tight way by increasing its contact surface with the discharge pipe and improving its adhesion. That is, the discharge pipe coupling hole may be configured such that the perforated hole is partially extended to increase the area of the inner wall surface and its inner diameter is slightly smaller than the discharge pipe to allow close adhesion when installed. Alternatively, a cushion layer of 1 to 3 mm may be coated on the inner surface of the discharge pipe coupling hole to strongly compress the discharge pipe, which may give water tightness.

The pressing plate 21 and the bottom plate 22 are preferably made of a rigid plate body such as plastic in order to maintain the shape of the storage container, and the sealing membrane 24 is preferably provided in the form of a rod that gives elasticity by being folded and unfolded according to the volume of the accommodation space therein. The sealing membrane may be used in a variety of forms, such as a stretchable form that expands or contracts due to its own material, a corrugated form that may be folded or unfolded in multiple stages, or a synthetic resin film that may be folded or unfolded freely.

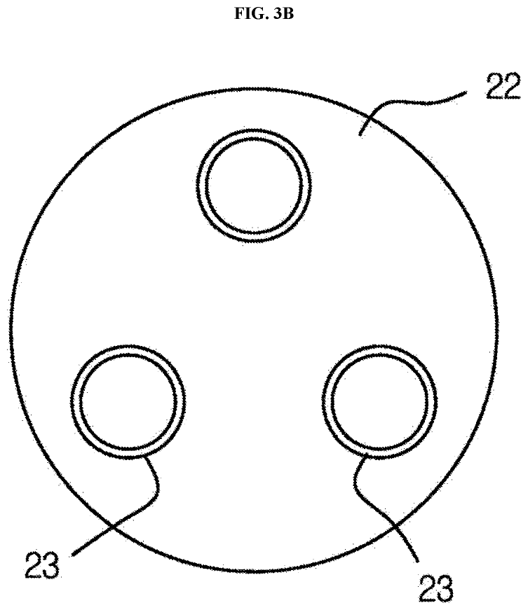

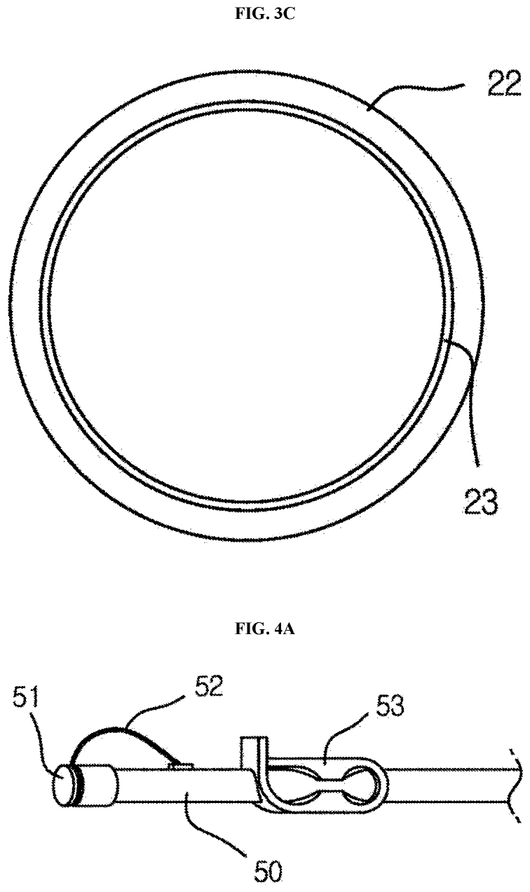

In addition, a spring 23 is further installed to the accommodation space. As shown in FIG. 3A, the spring 23 is interposed between the pressing plate 21 and the bottom plate 22 to give an elastic repulsive force such that the pressing plate and the bottom plate are pushed vertically, respectively. The spring 23 may be formed in a cylindrical shape to allow elastic deformation in the vertical direction, or formed in a triangular truncated cone shape whose winding has a smaller diameter upward, thereby minimizing the thickness of the folded portion when pressed by pressure.

Since the position of the spring 23 may be changed while the storage container 20 is folded or unfolded, a circular support wall 25 may protrude on opposite surfaces of the pressing plate 21 and the bottom plate 22 to fix the spring 23. That is, the circular support wall 25 protrudes downward on the bottom surface of the pressing plate 21, and the circular support wall 25 protrudes upward on the top surface of the bottom plate 22. Also, the diameter of the circular support wall 25 may be a little greater than the diameter of the spring 23 included therein, preventing the spring from being pushed out in a lateral direction. The circular support wall 25 preferably protrudes in the range of 3 to 10 mm from the surfaces of the pressing plate and the bottom plate to prevent the spring 23 from being pushed when the pressing force is applied thereto, thereby limiting lateral movement of the spring.

In addition, three springs 23 may be generally used to be arranged in a triangular formation as shown in FIG. 3B, or only one spring having a large diameter may be used to give an elastic force in a direction of pushing the pressing plate and the bottom plate from each other as shown in FIG. 3C.

In addition, in the existing technique, if only three springs are formed, the springs may be easily detached since the spring fixing portion is weak. However, in the present disclosure, since the springs are fixed by the circular support wall, it is possible to prevent the springs from being detached. In particular, if one large spring is installed, the large spring may be fixed by the circular support wall. Also, in the existing spring fitting configuration, the fitting area may be increased to secure firm fixation, thereby preventing the spring from being separated while being compressed and relaxed.

In this structure, the discharge pipe 50 is installed so that a portion thereof is located inside the storage container and the rest portion is exposed out of the storage container.

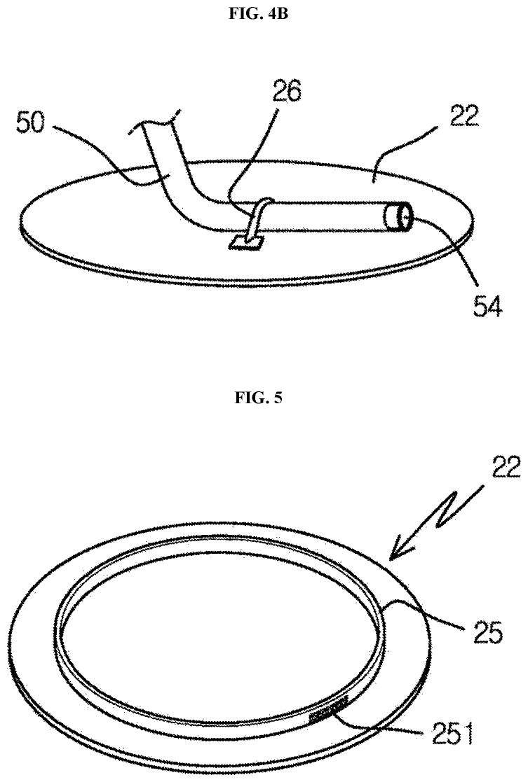

As shown in FIG. 4A, a stopper 51 for closing an end of the discharge pipe 50 is installed to the outwardly exposed portion of the discharge pipe 50, and the stopper may be connected to the discharge pipe by a stopper fixing string 52 to prevent the stopper from being lost during opening and closing.

Also, a clamp 53 is further installed on the line of the discharge pipe 50 to control the flow path of the discharge pipe. The clamp 53 is usually mounted to a medical tube to control the flow path, and the clamp 53 is coupled to an outer line of the discharge pipe to block the movement (discharge) of the body fluid stored in the storage container. In addition, if the function of the check valve does not work while the body fluid collected in the storage container is being discharged by pressing the pressing plate, the clamp may be manipulated to prevent the body fluid from flowing back into the storage container. In this way, it is possible to provide a double blocking function by the check valve and the clamp, such as prevention of infection.

As shown in FIG. 4B, a check valve 54 may be installed at an end of the discharge pipe 50 located inside the storage container such that the stored body fluid may be discharged out, while preventing back flow and inflow of external air. In addition, by using the discharge pipe fixing ring 26, the end of the discharge pipe 50 may be brought into close contact with the bottom plate 22 to absorb the body fluid at the bottom of the storage container and discharge the body fluid to the outside. The discharge pipe fixing ring 26 is provided such that its one end is coupled to the bottom plate and the other end covers an upper portion of the discharge pipe to be mounted to a bottom plate at an opposite side. Alternatively, in addition to the ring shape, a protrusion and a groove may be formed at the bottom of the discharge pipe and the bottom plate to face each other so as to be detachably coupled with each other. In this way, various known technologies may be applied.

In addition, the discharge pipe may be configured to be bent at various angles by forming a corrugated region in its portion exposed to the outside. The corrugated region may be provided by forming a plurality of folding portions in advance in a part of the discharge pipe so as to be stretched and extended or be bend in various directions. The corrugated region may be formed only in a certain region of the discharge pipe exposed to the outside, or in an entire region thereof or in multiple regions thereof.

In the above structure of the storage container 20, in a state where the pressing plate 21 and the bottom plate 22 are pressed in close contact with each other by pressure, if the pressure is removed, the storage container expands in an inflating direction by the restoring force of the spring 23 to form a negative pressure therein. At this time, since the discharge pipe 50 is closed by the check valve 54 installed at the lower end of the discharge pipe, the negative pressure is applied to the inlet pipe to inhale the body fluid of the human body and store the body fluid in the storage container.

In addition, in order to discharge the body fluid stored in the storage container 20, in a state where the stopper 51 installed at the end of the discharge pipe is opened and the clamp 53 is opened, the pressing plate 21 of the storage container is pressed to pressurize the inside. At this time, the check valve installed in the inlet pipe coupling hole 30 is closed to block the flow path of the inlet pipe, and the check valve 54 installed at the end of the discharge pipe 50 is opened to discharge the body fluid stored in the storage container to the outside through the discharge pipe 50.

If the discharge pipe 50 is installed to the aspirator 10 as described above, the stored body fluid is easily discharged without turning over the aspirator, so the body fluid discharge amount may be easily checked. In addition, since pressure is not transferred to the storage container while opening the stopper and the clamp installed to the discharge pipe, it is possible to prevent the body fluid from being ejected while opening the discharge pipe.

Since the aspirator 10 of the present disclosure discharges the stored body fluid without being turned over, it further includes a structure for easily discharging the stored body fluid.

For example, as shown in FIG. 5, the circular support wall 25 for supporting the spring 23 is formed on the bottom plate 22. Since the circular support wall separates the inside and the outside, if the discharge pipe is placed inside the circular support wall, the body fluid collected out of the circular support wall may not be easily discharged. Therefore, a plurality of perforated holes 251 may be formed in the circular support wall 25 formed on the bottom plate so that the body fluid collected inside or outside may flow and easily discharge by the discharge pipe. At this time, the perforated holes 251 may be formed on the entire surface of the circular support wall 25 or only at a side where the discharge pipe is placed. Also, the circular support wall itself may be formed by a plurality of protruding walls. That is, the circular support wall itself may be formed with a plurality of protruding walls, instead of a single wall, so that the body fluid located therein may flow outward through a gap between the protruding walls.

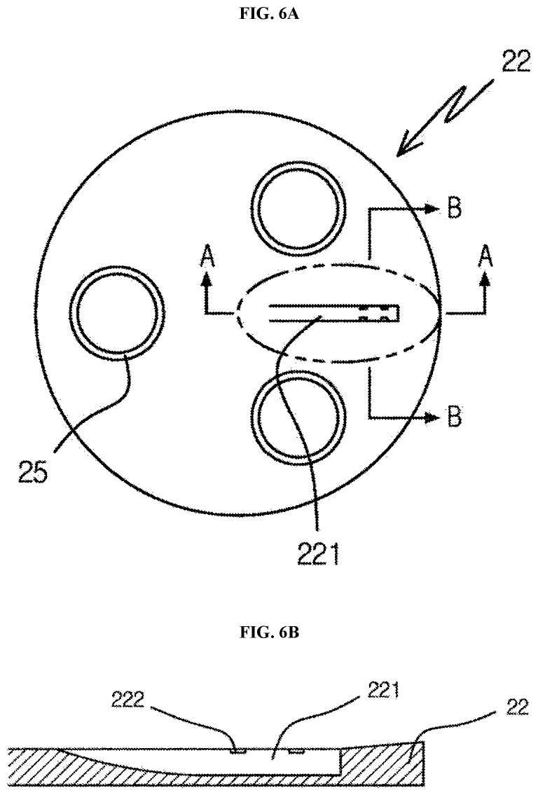

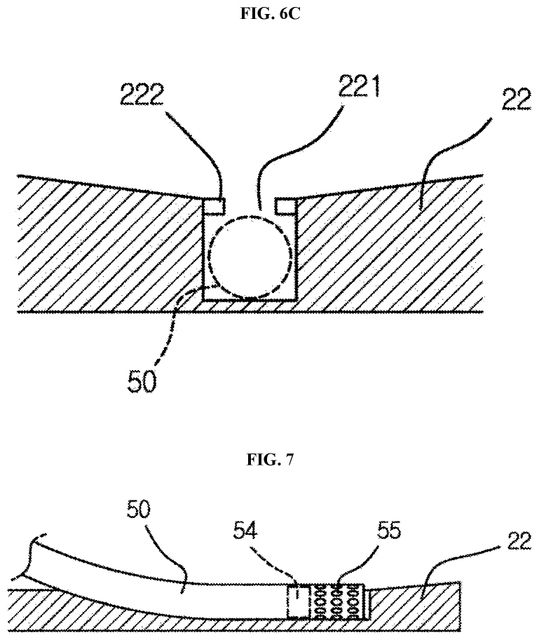

Next, as shown in FIGS. 6A to 6C, a placing groove 221 for placing the lower portion of the discharge pipe 50 on the bottom plate 22 may be further formed.

The placing groove 221 may be formed long in a longitudinal direction so that the shrinking movement of the discharge pipe required in the process of expanding and contracting the storage container is performed only within the placing groove. In this case, the end of the discharge pipe may be always positioned to perform inhaling at a regular part regardless of the expansion and contraction of the storage container.

In addition, the bottom plate 22 having the placing groove may have a downward slope from the edge toward the placing groove 221 so that the remaining body fluid may be moved to the placing groove without tilting the storage container in the process of discharging the stored body fluid.

In addition, as shown in FIG. 6C, the placing groove 221 may have a plurality of hanging protrusions 222 protruding on both sidewalls thereof so that the discharge pipe is fixed in a way of being fitted into the placing groove. If the end of the discharge pipe is fixed by the hanging protrusion 222, the discharge pipe fixing ring may not be additionally needed.

In addition, the placing groove may be formed to a variety of depths as required, for example to a depth in which the discharge pipe is entirely inserted or a depth in which the discharge pipe is inserted only partially.

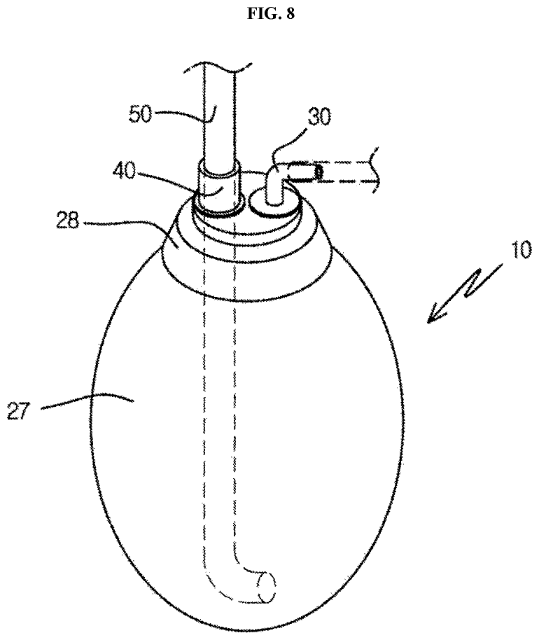

In addition, as shown in FIG. 7, a plurality of introduction holes 55 may be formed in a partial region of the lower end of the discharge pipe 50, so that the body fluid is introduced into the discharge pipe not only through then end of the discharge pipe but also through the introduction hole in the sidewall of the discharge pipe. In this case, even if the end of the discharge pipe is blocked by the sealing membrane or the inner wall of the placing groove, the body fluid may be easily sucked through the introduction hole in the sidewall. At this time, the check valve 54 installed at the end of the discharge pipe 50 may be formed in a region where the introduction hole 55 is not formed, so that the discharge pipe is controlled.

Meanwhile, FIG. 8 shows an aspirator according to another embodiment of the present disclosure.

As shown in the figure, the aspirator 10 according to another embodiment of the present disclosure may include a container portion 27 made of an oval or spherical body of an elastic material, and a cover portion 28 detachably coupled to an upper portion of the container portion and having the inlet pipe coupling hole 30 and the discharge pipe coupling hole 40.

The container portion 27 and the cover portion 28 may be formed in a detachable structure, and preferably, a screw connection method may be applied.

The inlet pipe coupling hole 30 and the discharge pipe coupling hole 40 described above are also formed in the cover portion 28, and the discharge pipe 50 is installed through the discharge pipe coupling hole 40 so that the lower end of the discharge pipe is disposed at the bottom surface of the inner side of the container portion to discharge the stored the body fluid as much as possible. Also, and a stopper and a clamp connected to a stopper fixing string may be installed at the upper portion of the discharge pipe located out of the container portion to control opening and closing of the flow path of the discharge pipe.

The aspirator 10 of this embodiment discharges the body fluid through the discharge pipe 50 by pressing and distorting the container portion 27 to change the internal volume thereof.

In addition, check valves may be formed at the inlet pipe coupling hole 30 and the discharge pipe 50, respectively, so that the external body fluid is introduced through the inlet pipe coupled to the inlet pipe coupling hole and the stored body fluid is discharged to the outside through the discharge pipe when pressure is applied thereto. This may prevent the inside of the aspirator from being contaminated by blocking the inflow of external air through the discharge pipe. The check valve is not limited to the above location and may be installed at any one of the inlet lines and the discharge line to control the flow of body fluid.

Hereinafter, an aspirator according to the second embodiment of the present disclosure will be described.

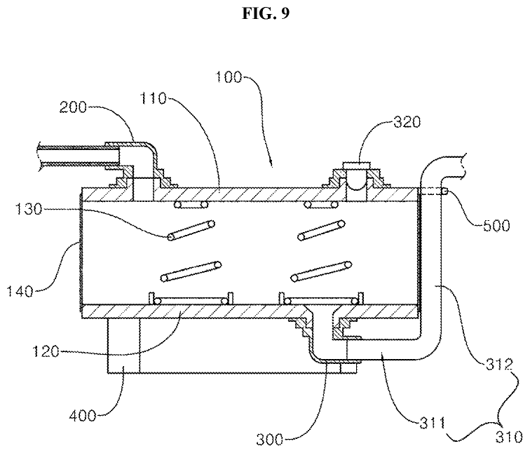

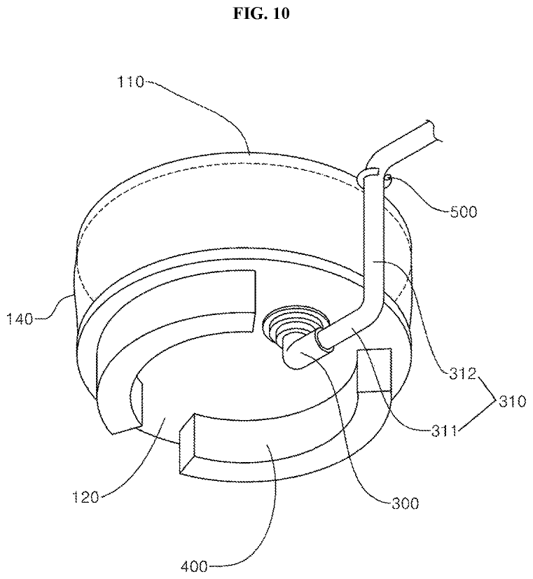

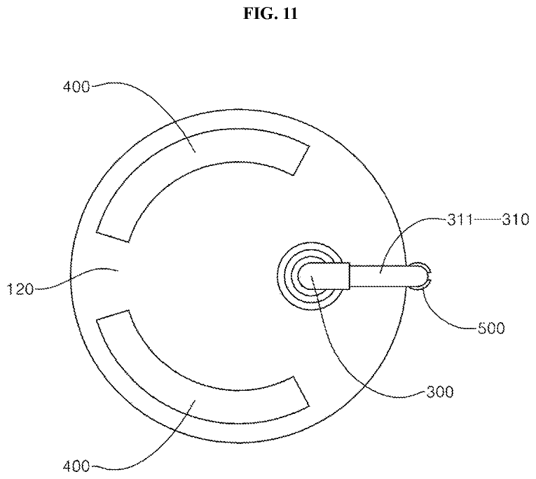

As shown in FIGS. 9 to 11, in the aspirator according to the second embodiment of the present disclosure, an inlet hole 200 for introducing a body fluid and a discharge hole 300 for discharging a stored body fluid are formed in the storage container 100 so that the discharge hole 300 is formed at the bottom plate 120 to protrude on a bottom surface of the storage container 100, a supporting protrusion 400 is further formed to protrude on the bottom surface of the bottom plate 120 to balance the storage container 100, and the discharge hole 300 further includes a discharge tube 310 extending to the outside of the storage container 100.

As described above, in the aspirator of the present disclosure, since the discharge hole 300 for discharging the body fluid is formed at the lower portion of the storage container 100 rather than the upper portion thereof, the discharged body fluid may be discharged through the discharge hole 300 located at the lower portion without turning over the storage container 100. At this time, an upper portion of an inner hole of the discharge hole 300 is preferably formed with a broad upper portion and a narrow lower portion so that its cross section has a “V” shape so that the body fluid is easily discharged.

The inlet hole 200 of the present disclosure is a portion through which the body fluid flows into the storage container 100. As shown in the figures, the inlet hole 200 may be formed at the upper portion of the storage container 100, namely at the pressing plate 110, as in a general case. However, the inlet hole 200 may also be formed to protrude downward at the bottom plate 120 in the same form as the discharge hole 300.

In addition, since the discharge hole 300 of the present disclosure formed at the lower portion protrudes on the bottom surface of the bottom plate 120, the storage container 100 may fall or tilt when it is placed in this state. However, since the supporting protrusion 400 is formed to protrude on the bottom surface of the bottom plate 120, the storage container 100 may be stably supported on the ground to maintain balance. Also, when a pressure is applied to the pressing plate 110 to discharge the body fluid, the discharge hole 300 and the discharge tube 310 are not pressed. Also, it is possible to prevent that the storage container 100 is not balanced and moves to the left and right to make it difficult for medical personnel to discharge the body fluid.

In addition, the discharge hole 300 of the present disclosure is not formed as a short hole as before but further includes a discharge tube 310 extending to the outside of the storage container 100. Thus, since the portion where the body fluid is discharged may be directly checked with the eye, the body fluid may be discharged conveniently. Also, since the direction in which the body fluid is discharged may be adjusted using the extending portion, there is no fear of fluid leakage and a hygienic effect is provided.

The discharge tube 310 is preferably made of a material that is easily bent, and the discharge tube 310 is shaped to include a horizontal connection portion 311 extending toward an edge of the bottom plate 120 and a vertical connection portion 312 bent vertically at an extension end of the horizontal connection portion 311 to extend upward at an outer side of the edge, as shown in the figures. At this time, a fixing member 500 is preferably formed at an outer side of the pressing plate 110 so that the vertical connection portion 312 is fixed to the edge of the pressing plate 110 to maintain a vertical state. Of course, even when the inlet hole 200 is formed to protrude at the lower portion of the bottom plate 120 in the same way as the discharge hole 300 of the present disclosure, the fixing member 500 of the same type may also be installed thereto.

Even without the fixing member 500, it is possible to discharge the body fluid through the discharge tube 310. However, since the discharge tube 310 may maintain a vertically extending shape without being dangled on the bottom surface of the bottom plate 120 by means of the fixing member 500, it is possible to prevent the interference problem of the discharge tube 310 and also prevent the phenomenon that the discharge tube 310 is pressed by the supporting protrusion 400.

In addition, the rest portion of the discharge tube 310 over the fixing member 500 may be freely bent or moved to control the direction in which the body fluid is discharged, as described above.

As shown in the figures, the fixing member 500 is formed to have a “C”-shaped ring form with one open side, so that the discharge tube 310 is inserted into and fixed to the open portion of the fixing member 500. Of course, in addition to the above, the discharge tube 310 may be fixed in various ways using a string, a clip or a pin, a Velcro tape, and the like.

Moreover, a valve (not shown) may be installed at the discharge tube 310 to open or close the movement of the body fluid. Since the valve may be formed in various shapes, its specific structure will not be described in detail.

The supporting protrusion 400 of the present disclosure is a component that is essentially formed as the discharge hole 300 is formed at the lower portion. That is, the supporting protrusion 400 serves to maintain the balance of the storage container 100 to be stably supported on the ground without falling, and is intended to prevent the discharge hole 300 from being pressed against the ground. If the discharge hole 300 is pressed against the ground while the body fluid is being discharged, the discharge tube 310 may be separated or the passage through which the body fluid moves may be narrowed, which may disturb smooth discharge.

Therefore, the supporting protrusion 400 of the present disclosure preferably protrudes further to a protrusion length of the discharge hole 300 so that the discharge hole 300 is spaced from the ground. Although the supporting protrusion 400 may gave any shape freely, its shape should not affect the path of the discharge hole 300 and the discharge tube 310 while supporting the storage container 100 as a whole.

Moreover, if the bottom surface of the bottom plate 120 has a groove shape in the portion where the discharge hole 300 and the discharge tube 310 are formed, the bottom surface of the bottom plate 120 excluding the groove portion may serve as the supporting protrusion 400 as a whole so that the discharge hole 300 and the discharge tube 310 are spaced from the ground.

In the present disclosure configured as above, the body fluid may be discharged entirely through the discharge hole 300 located at the lower portion of the storage container 100. However, an emergency discharge hole 320 may be further formed at the upper portion of the pressing plate 110 as shown in the figures so that the emergency discharge hole 320 may be used appropriately when the discharge hole 300 located at the lower portion is unavailable.

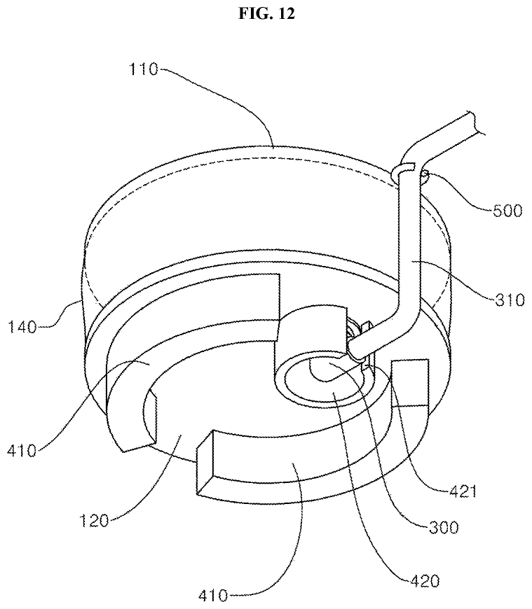

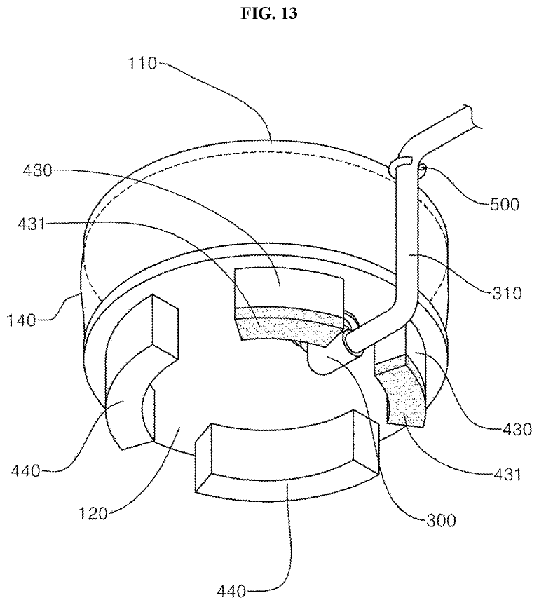

FIG. 12 shows another embodiment of the supporting protrusion 400 according to the present disclosure, which includes a main support portion 410 widely formed at the bottom surface to support the storage container 100 as a whole and maintain the balance of the storage container 100, and a sub support portion 420 formed only around the discharge hole 300 to prevent the discharge hole 300 from being deformed due to a pressure.

The sub support portion 420 is limitedly formed only to the portion where the discharge hole 300 is formed, and a cut portion 421 is preferably formed at the sub support portion 420 in a direction along which the discharge tube 310 is connected.

As described above, in this embodiment, since the sub support portion 420 is further formed, when the pressing plate 110 is pressed while the body fluid is being discharged, the discharge hole 300 and the discharge tube 310 may maintain their shapes without being deformed. Moreover, the sub support portion 420 may have an open bottom surface as shown in the figure, but may also be provided in the form of a cover to completely protect the discharge hole 300.

FIG. 13 shows another embodiment of the supporting protrusion 400 according to the present disclosure, which includes a front support portion 430 formed around the discharge hole 300 and a rear support portion 440 formed at a side opposite to the front support portion 430, wherein the front support portion 430 is made of an elastic material.

In this embodiment, when the pressing plate 110 is pressed while the body fluid is being discharged, the front support portion 430 made of an elastic material is further lowered while being compressed, and the rear support portion 440 made of a hard material maintains its location so that the storage container 100 is slightly tilted toward the front support portion 430. That is, since the front support portion 430 is located toward the discharge hole 300, the body fluid may be more easily discharged as the storage container 100 is tilted. The front support portion 430 made of an elastic material should be designed to have a sufficient protruding length so that the discharge hole 300 and the discharge tube 310 are not pressed against the ground even when the front support portion 430 is compressed by pressure.

The front support portion 430 may not be entirely made of an elastic material. That is, if an elastic member 431 made of a rubber material is coupled with a predetermined thickness to a portion of the front support portion 430 in contact with the ground as shown in the figure, as the storage container 100 is tilted, the body fluid may be easily discharged. Also, since the frictional force of the elastic member 431 is increased, the anti-slippery function may also be provided.

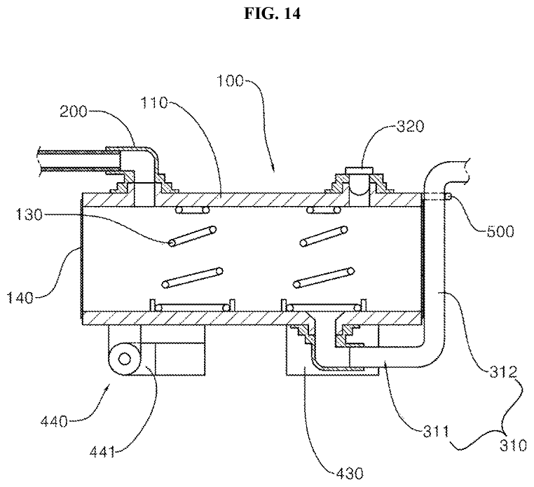

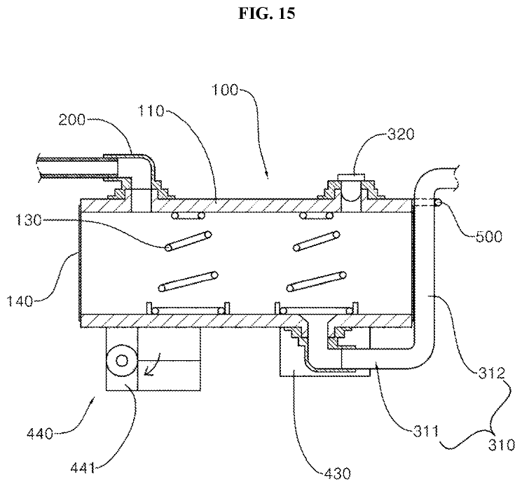

FIGS. 14 and 15 show another embodiment of the supporting protrusion 400 according to the present disclosure, which includes a front support portion 430 formed around the discharge hole 300 and a rear support portion 440 formed at a side opposite to the front support portion 430, wherein the rear support portion 440 is installed to have an adjustable support height.

That is, as shown in the figures, an extended support portion 441 capable of being be folded and unfolded by a hinge coupling is further formed at a lower portion of the rear support portion 440. The extended support portion 441 may be folded in parallel with the front support portion 430 at ordinary time as shown in FIG. 14, and when the body fluid is discharged, the extended support portion 441 is unfolded long as shown in FIG. 15 so that the storage container 100 is tilted toward the discharge hole 300 for easy discharge.

Of course, in order to enable the above function, the discharge hole 300 should be formed in a position eccentric to one side, as shown in the drawing, rather than a center of the bottom plate 120.

Hereinafter, a connection configuration of a drainage tube and a body fluid tube in an aspirator according to an embodiment of the present disclosure will be described.

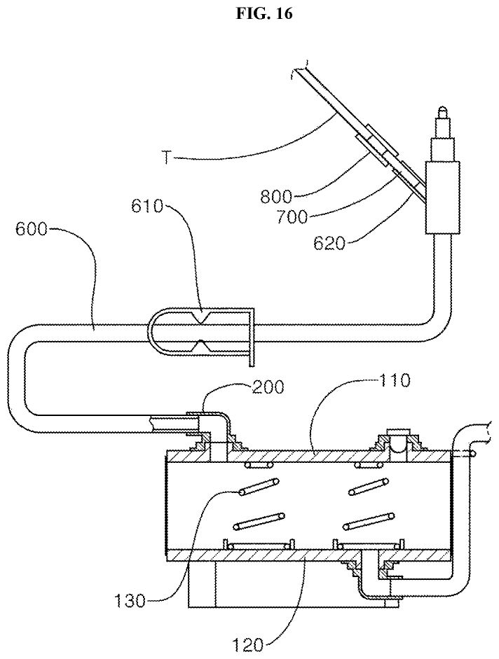

As shown in FIG. 16, in the connection configuration, the inlet hole 200 for introducing body fluid is formed in the pressing plate 110 or the bottom plate 120 of the storage container 100, and inlet hole 200 is connected to a soft drainage tube 600 having a clamp 610, and a connection portion 620 is formed at the drainage tube 600 so that a soft body fluid tube T fixed to the body of a patient is connected thereto.

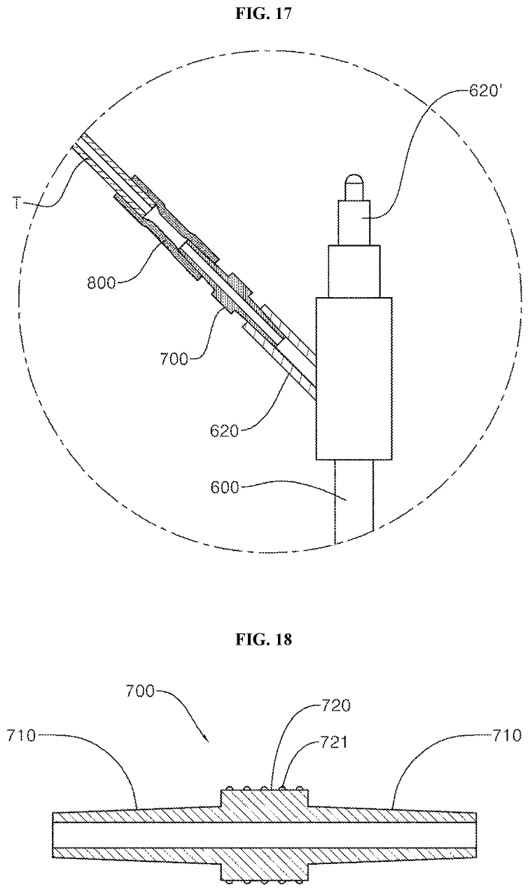

Here, as shown in an enlarged view of FIG. 17, the present disclosure has a characteristic in that a hard primary connection tube 700 is firstly coupled to the connection portion 620 between the connection portion 620 of the drainage tube 600 and the body fluid tube T, and a soft secondary connection tube 800 is coupled to the primary connection tube 700 so as to be connected to the body fluid tube T.

That is, the primary connection tube 700 is coupled to be inserted into the secondary connection tube 800, and the secondary connection tube 800 is coupled to surround the outer circumference of the body fluid tube T.

The primary connection tube 700 is made of a rigid material such as plastic. Both sides of the primary connection tube 700 are inserted into the connection portion 620 of the soft drainage tube 600 and the secondary connection tube 800, respectively, and allows easy coupling because hard material and soft material are coupled.

Moreover, the primary connection tube 700 and the secondary connection tube 800 of the present disclosure may also be connected to an auxiliary connection portion 320′ formed at another location than the connection portion 620 basically used in the drainage tube 600. The auxiliary connection portion 320′ is a known component that is also formed at a conventional drainage tube, and if required, the auxiliary connection portion 320′ may be cut so that another body fluid tube T is connected thereto.

Seeing the configuration of the primary connection tube 700, as shown in FIG. 18, fitting portions 710 are formed at both sides thereof so that the connection portion 620 and the secondary connection tube 800 are coupled thereto, respectively, and a handle portion 720 protruding further to the outer diameter of the fitting portion 710 is formed between the fitting portions 710 at both sides.

The handle portion 720 has a protruding configuration to facilitate the insertion process by the user, and an anti-slip protrusion 721 may be further formed on the surface of the handle portion 720.

In addition, the fitting portion 710 preferably has an outer diameter larger than the inner diameter of the connection portion 620 and the secondary connection tube 800, so as to have a strong coupling force upon coupling. Also, more preferably, as shown in the figures, if the fitting portion 710 is inclined such that its outer diameter gradually increases along the insertion direction, the insertion process may be more smoothly performed.

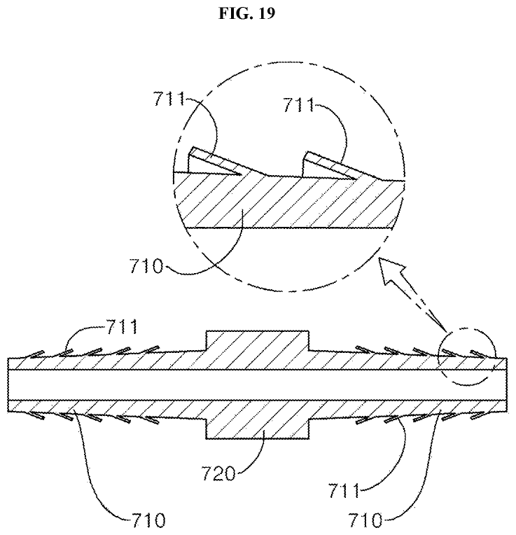

Moreover, as shown in FIG. 19, if the fitting portion 710 further includes a first wedge portion 711 having a plurality of protrusions tapered at a certain angle on the outer circumference thereof along a longitudinal direction, the coupling force may be further increased. That is, since the first wedge portion 711 is made of a hard material similar to the primary connection tube 700, the first wedge portion 711 is strongly adhered to the inside of the soft connection portion 620 and the secondary connection tube 800. Also, since the first wedge portion 711 is inclined along the insertion direction, the first wedge portion 711 may be inserted easily but is not easily separated during the separation process since the inclined direction is opposite to the separation direction.

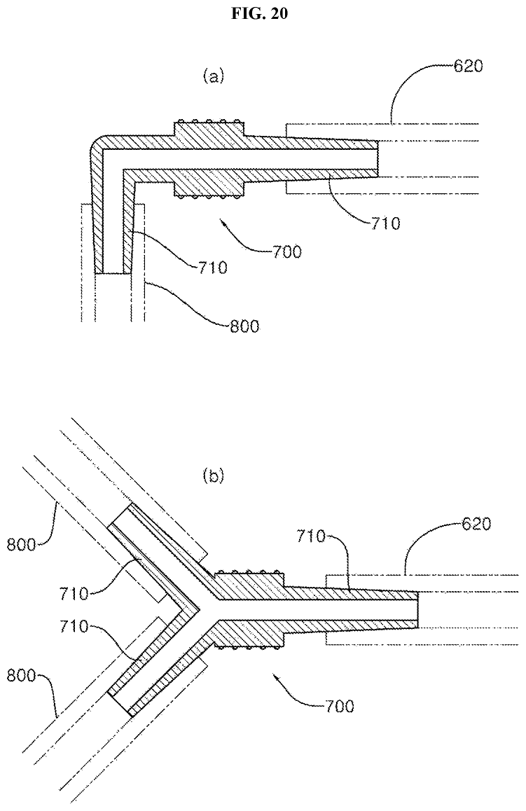

Moreover, the primary connection tube 700 of the present disclosure may also be variously shaped as shown in FIG. 20. As shown in (A) of FIG. 20, the fitting portion 710 of the primary connection tube 700 may be configured to be bent at a certain angle so that the coupling may be more easily performed. At this time, the bent angle may be variously performed, such as 30 degrees, 60 degrees and 90 degrees. In addition, as shown in (B) of FIG. 20, the fitting portion 710 may be provided at two places in the direction along which the secondary connection tube 800 is coupled to the primary connection tube 700 so as to form a “Y” shape. This configuration gives an effect that two body fluid tubes T may be connected to one connection portion 620.

The secondary connection tube 800 of the present disclosure is made of a soft material so that the primary connection tube 700 is inserted at one side and the body fluid tube T is inserted at the other side. The inner diameter of the secondary connection tube 800 is preferably smaller than the outer diameter of the primary connection tube 700 and the body fluid tube T so that the coupled portion has a strong fastening force.

The secondary connection tube 800 of the present disclosure is made of a soft material as above, but the inner diameter into which the body fluid tube T is inserted and the material strength may be changed according to the type of the body fluid tube T. The drainage tube 600 is made of one material from the part connected to the storage container to the connection portion 620, so it is difficult to immediately change the design depending on the characteristics of the body fluid tube T. However, the secondary connection tube 800 of the present disclosure may be redesigned to have a size optimized for the outer diameter of the body fluid tube T, and the material of the secondary connection tube 800 may be easily adjusted in consideration of the material strength of the body fluid tube T. For this reason, the coupling force may be enhanced.

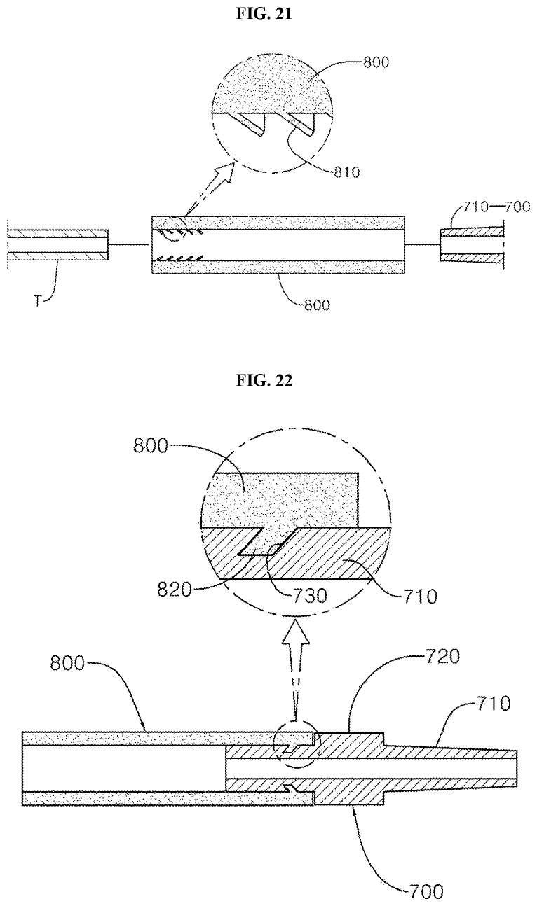

In addition, as shown in FIG. 21, in order to further increase the coupling force between the secondary connection tube 800 and the body fluid tube T, a second wedge portion 810 having a plurality of protrusions tapered at a certain angle along the longitudinal direction may be further provided at an inner side of the secondary connection tube 800 in a direction in which the body fluid tube T is inserted. That is, since the second wedge portion 810 is also made of a soft material similar to the secondary connection tube 800, the second wedge portion 810 is bent as the body fluid tube T is inserted, and thus comes into in strong contact with the body fluid tube T. Also, since the second wedge portion 810 is inclined in the insertion direction, the second wedge portion 810 may be inserted easily but is not easily separated during the separation process since the inclined direction is opposite to the separation direction.

In addition, in order to further prevent the separation of the secondary connection tube 800 from the body fluid tube T, the outer side of the secondary connection tube 800 to which the body fluid tube T is coupled may be further fixed using a separate means such as a thread, a string, a clip and a Velcro tie.

Moreover, the primary connection tube 700 and the secondary connection tube 800 of the present disclosure may be used to be coupled in a sequential manner at a medical site. However, as shown in FIG. 22, the primary connection tube 700 and the secondary connection tube 800 may be provided in an integrally combined form so as to be used directly at a medical site without the process of combining the primary connection tube 700 and the secondary connection tube 800.

In order to configure the primary connection tube 700 and the secondary connection tube 800 in an integrally combined form as described above, as shown in the figure, a coupling groove 730 may be formed at a predetermined depth on the outer circumference of the fitting portion 710 of the primary connection tube 700, and a coupling protrusion 820 shaped corresponding to the coupling groove 730 may be formed at the inner side of the secondary connection tube 800, such that the coupling groove 730 and the coupling protrusion 820 are coupled to each other.

At this time, in order to prevent the coupling groove 730 and the coupling protrusion 820 from being separated, the coupling groove 730 and the coupling protrusion 820 are preferably formed in an inclined shape in the insertion direction.

Moreover, in order to further increase the coupling force between the coupling groove 730 and the coupling protrusion 820, an adhesive material (not shown) may be firstly applied between the coupling groove 730 and the coupling protrusion 820, and then the coupling groove 730 and the coupling protrusion 820 may be coupled later.

Even though the present disclosure has been described with reference to the embodiment, various modifications can be made within the technical scope of the present disclosure.

1. A medical aspirator with improved safety, which receives and temporarily stores human body fluid, the medical aspirator comprising:
a storage container having an accommodation space formed therein;
an inlet pipe coupling hole formed at an upper portion of the storage container so that an inlet pipe for introducing human body fluid is mounted thereto;
a discharge pipe coupling hole formed at the upper portion of the storage container to communicate an inside and an outside of the storage container;
a discharge pipe coupled to the discharge pipe coupling hole and having a lower end located at a bottom of the accommodation space of the storage container and an upper end drawn out long to discharge body fluid stored in the storage container; and
a fixing means configured to fix the discharge pipe to the bottom of the accommodation space of the storage container.

2. The medical aspirator with improved safety of claim 1, wherein the discharge pipe drawn out includes a stopper provided at the upper end of the discharge pipe to close the discharge pipe, a stopper fixing string configured to connect the stopper to the discharge pipe, and a clamp mounted to an intermediate portion of the discharge pipe to control opening and closing of a flow path therein by pressure.

3. The medical aspirator with improved safety of claim 2, wherein the discharge pipe includes a check valve installed at the lower end thereof, which is located inside the storage container, to prevent back flow.

4. The medical aspirator with improved safety of claim 1, wherein the storage container comprising:
a pressing plate having an inlet pipe coupling hole and a discharge pipe coupling hole formed therein and having a circular support wall formed at a bottom surface thereof to protrude downward;
a bottom plate having a circular support wall formed at an upper surface thereof to protrude upward;
a spring disposed at an inner side of the circular support wall between the pressing plate and the bottom plate so that the pressing plate and the bottom plate are separated from each other; and
a sealing membrane configured to connect edges of the pressing plate and the bottom plate so that an accommodation space is formed therein.

5. The medical aspirator with improved safety of claim 4, wherein three springs are arranged at equal angles, three circular support walls supporting the springs are formed at the pressing plate and the bottom plate, respectively, so that ends of the springs are partially placed and fixed thereon, and a plurality of perforated holes are formed in a wall surface at one side of the circular support wall formed at the bottom plate so that the body fluid collected inside the circular support walls are discharged out of the circular support walls through the perforated holes.

6. The medical aspirator with improved safety of claim 4, wherein one spring is formed to have a diameter approximate to an inner surface of the sealing membrane, one circular support wall supporting the spring is formed at the pressing plate and the bottom plate, respectively, so that an end of the spring is partially placed and fixed thereon, and a plurality of perforated holes are formed in a wall surface at one side of the circular support wall formed at the bottom plate so that the body fluid blocked by the circular support wall not to move is moved into and out of the circular support wall through the perforated holes and discharged through the discharge pipe.

7. The medical aspirator with improved safety of claim 4, wherein the bottom plate has a placing groove formed long in a longitudinal direction at an upper surface thereof, and an edge of the bottom plate is configured to be inclined downward toward the placing groove so that the body fluid introduced into the storage container is collected in the placing groove.

8. The medical aspirator with improved safety of claim 4, wherein the fixing means includes a discharge pipe fixing ring having one end coupled to the bottom plate and the other end covering an upper portion of an end of the discharge pipe so that the other end is mounted to the bottom plate at an opposite side to fix the discharge pipe to the bottom, or includes a placing groove formed long in a longitudinal direction at an upper surface of the bottom plate and a plurality of hanging protrusions formed at an upper portion of a sidewall of the placing groove to protrude so that the discharge pipe is fitted into and fixed to the placing groove.

9. The medical aspirator with improved safety of claim 1, wherein the storage container comprising:
a container portion having an oval or spherical shape with an elastic material; and
a cover portion detachably coupled to the upper portion of the container portion and having an inlet pipe coupling hole to which the inlet pipe is mounted and a discharge pipe coupling hole to which the discharge pipe is mounted.

10. The medical aspirator with improved safety of claim 1, wherein a corrugated region is formed in a portion of the discharge pipe that is exposed to the outside so that the discharge pipe is bent at various angles.

11. A medical aspirator with improved safety, which includes a storage container having a pressing plate and a bottom plate respectively provided to an upper portion and a lower portion thereof and having a storage space formed by a sealing membrane that connects the pressing plate and the bottom plate, and a spring installed between the pressing plate and the bottom plate, wherein the improvement comprises:
an inlet hole for introducing body fluid and a discharge hole for discharging stored body fluid are formed at the storage container, and the discharge hole is formed at the bottom plate to protrude on a bottom surface of the storage container; and
a supporting protrusion is further formed to protrude on the bottom surface of the bottom plate for balancing the storage container,
wherein the discharge hole further includes a discharge tube configured to extend out of the storage container.

12. The medical aspirator with improved safety of claim 11, wherein the discharge tube includes a horizontal connection portion configured to extend toward a rim of the bottom plate and a vertical connection portion vertically bent at an extension end of the horizontal connection portion to extend upward at an outer side of the rim, and further includes a fixing member fixed to the rim of the pressing plate at an outer side of the pressing plate to maintain a vertical state the vertical connection portion.

13. The medical aspirator with improved safety of claim 12, wherein the fixing member is in the form of a “C”-shaped ring with one open side so that the discharge tube (310) is fit into and fixed to the open portion of the fixing member.

14. The medical aspirator with improved safety of claim 11, wherein the supporting protrusion protrudes further to a protruding length of the discharge hole so that the discharge hole is spaced apart from the ground.

15. The medical aspirator with improved safety of claim 11, wherein the supporting protrusion includes a main support portion formed broadly at a bottom surface thereof to balance the storage container, and a sub support portion formed around the discharge hole to prevent the discharge hole from being deformed due to pressure.

16. A medical aspirator with improved safety, which includes a storage container having a pressing plate and a bottom plate respectively provided to an upper portion and a lower portion thereof and having a storage space formed by a sealing membrane that connects the pressing plate and the bottom plate, and a spring installed between the pressing plate and the bottom plate, wherein the improvement comprises:
an inlet hole for introducing body fluid is formed at the pressing plate or the bottom plate of the storage container, a soft drainage tube having a clamp is connected to the inlet hole, and a connection portion is formed at the drainage tube so that a soft body fluid tube fixed to the body of a patient is connected thereto; and
a hard primary connection tube is firstly coupled to the connection portion between the connection portion and the body fluid tube, and a soft secondary connection tube is coupled to the primary connection tube so as to be connected to the body fluid tube.

17. The medical aspirator with improved safety of claim 16, wherein the primary connection tube is coupled so as to be inserted into the secondary connection tube, and the secondary connection tube is coupled so as to surround an outer circumference of the body fluid tube.

18. The medical aspirator with improved safety of claim 16, wherein the primary connection tube has fitting portions formed at both sides thereof so that the connection portion and the secondary connection tube are respectively coupled thereto, and a handle portion protruding further to an outer diameter of the fitting portion is formed between both fitting portions.

19. The medical aspirator with improved safety of claim 18, wherein the fitting portion is inclined such that an outer diameter thereof is gradually increasing along the inserting direction.

20. The medical aspirator with improved safety of claim 18, wherein a first wedge portion having a plurality of protrusions tapered at a predetermined angle and formed along a longitudinal direction are further provided at the outer circumference of the fitting portion.

21. The medical aspirator with improved safety of claim 16, wherein the secondary connection tube further includes a second wedge portion formed at an inner side thereof at which the body fluid tube is inserted, the second wedge portion having a plurality of protrusions tapered at a predetermined angle and formed along a longitudinal direction.
